(19) United States
(12) Patent Application Publication
(10) Pub. No.: US 2026/0256621 A1
(43) Pub. Date: Sep. 3, 2026

(54) EYE MASK

(71) Applicant: RELIVE CO., LTD., Sendai-shi (JP)

(72) Inventor: Takashi SASAKI, Sendai-shi (JP)

(73) Assignee: RELIVE CO., LTD., Sendai-shi (JP)

(21) Appl. No.: 19/540,996

(22) Filed: Feb. 16, 2026

Related U.S. Application Data
(63) Continuation of application No. PCT/JP2024/028833, filed on Aug. 9, 2024.

(30) Foreign Application Priority Data
Aug. 17, 2023 (JP) 2023-133171
Oct. 30, 2023 (WO) PCT/JP2023/039165

(51) Int. Cl. A61F 9/04

(52) U.S. Cl. CPC A61F 9/04

(57) ABSTRACT

Resolution Means: An eye mask 100 of the present invention comprises an eye-mask body piece 10 for covering the eyes, and mineral-containing sites 50 situated in the eye-mask body piece 10. The eye-mask body piece 10 includes a frontward mask portion 11 for covering the eyes, and sideward mask portions 12, wherein among the mineral-containing sites 50 a frontward mineral patterning 51 for enveloping the periphery of the eyes is formed on the frontward mask portion 11. The frontward mineral patterning 51 is made up of two openings 53, and loop sites 54 positioned on the peripheries of the two openings 53. The loop sites 54 are created by a single strand of tape material being folded over itself.

### TECHNICAL FIELD

The present invention relates to eye masks. It especially relates to eye masks for alleviating fatigue in the wearer.

For the record, the present application claims right of priority based on Japanese Pat. App. No. 2023-133171, filed Aug. 17, 2023; the entire contents of that application are incorporated into the present specification by way of reference. The present application further claims right of priority based on International Pat. App. PCT/JP 2023/39165, filed Oct. 30, 2023; the entire contents of that application are incorporated into the present specification by way of reference.

### BACKGROUND ART

To date, an eye mask rendered with built-in masses of tourmaline particulates so as to give forth far infrared rays and negative ions in order to obtain relaxation benefits and fatigue-recovery effects has been proposed. Yet with this sort of eye mask is the drawback that because the tourmaline particulate masses are housed within eye covers rendered into pouch form, the negative ions given forth by the tourmaline are not readily released to the eye-mask exterior, burdening the weight of the eye mask (reference is made to Patent Document 1).

To ameliorate this sort of drawback, an eye mask such as presented in Patent Document 1 has been proposed. Specifically, the eye mask set forth in Patent Document 1 is configured with an eye-mask body into which ceramic plates that give off far-infrared rays have been built in positions opposing either eye, and negative-ion-generating sheets are fitted detachably/reattachably overlapping the reverse side of the eye-mask body. The negative-ion-generating sheets are constituted from an elastically extensible/contractable thin membranous material, and are formed with a mineral powder of high-level negative-ion-generating efficiency mixed in. According to the eye mask of Patent Document 1, the synergistic effect of the thermal efficacy of the far-infrared rays and of the benefits of the negative ions can alleviate eye fatigue, and make the healthiness of the eyes, and the skin and the skin tissue surrounding them, better.

### PRECEDENT TECHNICAL LITERATURE

### Patent Documents

Patent Document 1: Japanese Laid-Open Pat. Pub. No. 2003-310654.

### SUMMARY OF INVENTION

### Issues Invention is to Resolve

The present-application inventors, rather than simply around the eyes arranging objects in which tourmaline particulate masses are housed, or arranging negative-ion-generating sheets, in a different approach from that, arrived at the present invention taking on the challenge of developing an eye mask that alleviates fatigue in the wearer.

A principal object of the present invention, which has been brought about taking such points into consideration, is to make available an eye mask that alleviates fatigue in the wearer.

### Means for Resolving the Issues

An eye mask involving the present invention comprises an eye-mask body piece for covering the eyes, and mineral-containing sites situated in the eye-mask body piece and containing a mineral(s). The eye-mask body piece is constituted from a frontward mask portion for covering the eyes, and sideward mask portions extending from the frontward mask portion. On the frontward mask portion of the eye-mask body piece, among the mineral-containing sites a frontward mineral patterning for enveloping the periphery of the eyes is formed. The frontward mineral patterning is made up of two openings, and loop sites positioned on the peripheries of the two openings. The loop sites are created by a single strand of tape material being folded over itself.

In a preferred embodying mode, the loop sites have a figure-eight form.

In a preferred embodying mode, the single strand of tape material constituting the loop sites crosses over itself in a location situated in the middle between the two openings.

With a preferred embodying mode, in the loop sites, by the single strand of tape material being folded over itself, sites where the obverse face and reverse face of the single strand of tape material overlap each other are present in at least seven locations.

With a preferred embodying mode, the loop sites have the form of a figure eight having a Möbius-band structure.

In a preferred embodying mode, the two openings in the frontward mineral patterning are eye-corresponding openings that correspond to eye areas.

In a preferred embodying mode, on the eye-mask body piece a first patch constituted from a first metal is formed, wherein separated from the first patch a second patch constituted from a second metal then is formed.

In a preferred embodying mode, the first patch is a gold patch, and therein the second patch is a silver patch.

In a preferred embodying mode, the two openings in the frontward mineral patterning are eye-corresponding openings that correspond to the eye areas. The first patch is provided on at least a part of the loop sites or the openings that corresponds to one of either the right eye or the left eye. The second patch is provided on at least a part of the loop sites or the openings that corresponds to the other than the one of either the right eye or the left eye.

In a preferred embodying mode, the first patch is provided in the loop sites in the frontward mineral patterning, and the second patch is provided in at least one region of the two openings in the frontward mineral patterning.

In a preferred embodying mode, the first patch is provided in the loop sites in the frontward mineral patterning. The second patch is provided in the loop sites in the frontward mineral patterning.

In a preferred embodying mode, the first patch and the second patch are sewn on, anchoring them to the loop sites.

In a preferred embodying mode, only a portion of the first patch is provided on the loop sites.

In a preferred embodying mode, only a portion of the second patch is provided on the loop sites.

In a preferred embodying mode, on the sideward mask portions of the eye-mask body piece, among the mineral-containing sites sideward mineral patternings that extend along the lateral sides of the head are formed.

In a preferred embodying mode, the sideward mineral patternings are made up of rectilinear patterns.

In a preferred embodying mode, the mineral is at least one type, or a mixture of a plurality of mineral types, selected from the group consisting of radium ore, germanium, quartz, “terahertz” ore, and tourmaline. The mineral(s) is invested into the tape material by being printed onto it. The mineral(s) invested by the printing have a pattern form selected from the group consisting of geometric shapes, design marks, and written characters.

In a preferred embodying mode, the mineral-containing sites are disposed in the interior of the eye-mask body piece.

An eye mask involving the present invention comprises an eye-mask body piece for covering the eyes, and mineral-containing sites situated in the eye-mask body piece and containing a mineral(s). The eye-mask body piece is constituted from a frontward mask portion for covering the eyes, and sideward mask portions extending from the frontward mask portion. On the frontward mask portion of the eye-mask body piece, among the mineral-containing sites a frontward mineral patterning for enveloping the periphery of the eyes is formed. The frontward mineral patterning is made up of two openings, and loop sites positioned on the peripheries of the two openings. On the eye-mask body piece a first patch constituted from a first metal is formed. Separated from the first patch, a second patch constituted from a second metal then is formed.

In a preferred embodying mode, the first patch is a gold patch. The second patch then is a silver patch.

In a preferred embodying mode, the two openings in the frontward mineral patterning are eye-corresponding openings that correspond to the eye areas. The first patch is provided on at least a part of the loop sites or the openings that corresponds to one of either the right eye or the left eye. The second patch is provided on at least a part of the loop sites or the openings that corresponds to the other than the one of either the right eye or the left eye.

An eye-mask manufacturing method involving the present invention includes a step of forming on a frontward mask portion of an eye-mask body piece for covering the eyes a frontward mineral patterning for enveloping the periphery of the eyes. The frontward mineral patterning is formed by affixing a mineral tape material in which a mineral(s) has been invested into the tape material by being printed onto it. The mineral is at least one type, or a mixture of a plurality of mineral types, selected from the group consisting of radium ore, germanium, quartz, “terahertz” ore, and tourmaline. In the step of forming the frontward mineral patterning, the formation of the frontward mineral patterning is executed in such a way as to create by a single strand of the tape material being folded over itself eye-corresponding openings that correspond to eye areas.

With a preferred embodying mode, in the step of forming the frontward mineral patterning, the creating of loop sites having a figure-eight form by folding the single strand of the tape material over itself is executed.

With a preferred embodying mode, in the folding of a single strand of tape material over itself, the folding-over is executed in such a way that sites where the obverse face and reverse face of the single strand of the tape material overlap each other are present in at least seven locations.

With a preferred embodying mode, in the step of forming the frontward mineral patterning, the creating of regions in figure-eight form having a Möbius-band structure by folding the single strand of the tape material over itself is executed.

With a preferred embodying mode, further, a step of creating sideward mineral patternings in such a way as to be adjacent to or contiguous with the frontward mineral patterning is executed.

With a preferred embodying mode, on the eye-mask body piece a step of creating a first patch constituted from a first metal, and a step of creating a second patch, separated from the first patch, constituted from a second metal are executed.

A corporeal contact article involving the present invention is furnished with a corporeal-contact body section for covering a portion of the body, and mineral-containing sites situated in the corporeal-contact body section and containing a mineral(s). The mineral-containing sites are configured through two openings, and loop sites positioned on the peripheries of the two openings. The loop sites are created by a single strand of tape material being folded over itself.

In a preferred embodying mode, the loop sites have a figure-eight form.

In a preferred embodying mode, the single strand of tape material constituting the loop sites crosses over itself in a location situated in the middle between the two openings.

With a preferred embodying mode, in the loop sites, by the single strand of tape material being folded over itself, sites where the obverse face and reverse face of the single strand of tape material overlap each other are present in at least seven locations.

In a preferred embodying mode, the loop sites have the form of a figure eight having a Möbius-band structure.

In a preferred embodying mode, the mineral is at least one type, or a mixture of a plurality of mineral types, selected from the group consisting of radium ore, germanium, quartz, “terahertz” ore, and tourmaline. The mineral(s) is invested into the tape material by being printed onto it. The mineral(s) invested by the printing have a pattern form selected from the group consisting of geometric shapes, design marks, and written characters.

In a preferred embodying mode, on the mineral-containing sites a first patch constituted from a first metal is formed, wherein separated from the first patch a second patch constituted from a second metal then is formed. The first patch is provided in a region of the loop sites in the mineral-containing sites, and the second patch is provided in at least one region of the two openings in the mineral-containing sites.

In a preferred embodying mode, for the mineral-containing sites, further, extension mineral patternings that extend adjacent to or contiguous with the loop sites are provided.

In a preferred embodying mode, the corporeal contact article is a support article for protecting at least one of either an elbow or a knee.

In a preferred embodying mode, the corporeal contact article is disposed within a necktie.

An eye mask involving an embodying mode of the present invention comprises an eye-mask body piece for covering the eyes, and mineral-containing sites situated in the eye-mask body piece and containing a mineral, with the eye-mask body piece being constituted from a frontward mask portion for covering the eyes, and sideward mask portions extending from the frontward mask portion. On the frontward mask portion of the eye-mask body piece, among the mineral-containing sites a frontward mineral patterning for enveloping the periphery of the eyes is formed. The frontward mineral patterning is configured through two openings, and loop sites positioned on the peripheries of the two openings. The loop sites have the form of a figure eight having a Möbius-band structure.

In a preferred embodying mode, on the eye-mask body piece a first patch constituted from a first metal is formed, wherein separated from the first patch a second patch constituted from a second metal then is formed, with the first patch being provided in the loop sites in the frontward mineral patterning, and the second patch being provided in at least one region of the two openings in the frontward mineral patterning.

A corporeal contact article involving an embodying mode of the present invention is furnished with a corporeal-contact body section for covering a portion of the body, and mineral-containing sites situated in the corporeal-contact body section and containing a mineral(s), wherein the mineral-containing sites are configured through two openings, and loop sites positioned on the peripheries of the two openings. The loop sites have the form of a figure eight having a Möbius-band structure.

In a preferred embodying mode, on the mineral-containing sites a first patch constituted from a first metal is formed, wherein separated from the first patch a second patch constituted from a second metal then is formed, with the first patch being provided in a region in the loop sites in the mineral-containing sites, and the second patch being provided in at least one region of the two openings in the mineral-containing sites.

### Effects of Invention

With an eye mask of the present invention, among mineral-containing sites situated in an eye-mask body piece for covering the eyes, two openings and loop sites are formed in a frontward mineral patterning, thanks to which when the eye mask is worn, fatigue in the wearer can be alleviated.

### BRIEF DESCRIPTION OF DRAWINGS

FIG. 1: A perspective view schematically representing, along the front, the configuration of an eye mask 100 involving modes of embodying the present invention.

FIG. 2: A perspective view schematically representing, along the back, the configuration of the eye mask 100.

FIG. 3: A front elevation schematically representing the configuration of the eye mask 100.

FIG. 4: A rear elevation schematically representing the configuration of the eye mask 100.

FIG. 5: A top-side view schematically representing the configuration of the eye mask 100.

FIG. 6: A bottom-side view schematically representing the configuration of the eye mask 100.

FIG. 7: A right-side elevation schematically representing the configuration of the eye mask 100.

FIG. 8: A left-side elevation schematically representing the configuration of the eye mask 100.

FIG. 9: A diagram for explaining the configuration of mineral-containing sites in the eye mask 100.

FIG. 10: A diagram schematically representing a tape article 20 of present embodying modes.

FIG. 11: An enlarged view for explaining the configuration of a frontward mineral patterning 51 of the mineral-containing sites 50.

FIG. 12: A diagram illustrating muscles 91 through 94 in the back side (nape) of the head portion 90.

FIG. 13: A diagram for explaining the head (90) skeletal structure (skull).

FIG. 14: FIG. 14A and FIG. 14B are views schematically representing, respectively, states in which the skull is dilated (expanded) and constricted (contracted).

FIG. 15: A diagram for explaining another configuration (modified example) of mineral-containing sites 50 in the eye mask 100.

FIG. 16: An enlarged diagram for explaining the configuration of a frontward mineral patterning 51.

FIG. 17: A diagram for explaining the relationship (flow of magnetism) between a first patch 61 and a second patch 62.

FIG. 18: A diagram for explaining a separate way of wearing an eye mask 100.

FIG. 19: A diagram for explaining a state in which an eye mask 100 has been packed compactly.

FIG. 20: A table setting forth different indicators in evaluations when the eye mask 100 was worn.

FIG. 21: A table setting forth test results (evaluation results) when the eye mask was worn.

FIG. 22: A perspective view schematically representing the configuration of a corporeal contact article (elbow support) 101 involving a mode of embodying the present invention.

FIG. 23: A perspective view schematically representing the configuration of a corporeal contact article (knee support) 102 involving a mode of embodying the present invention.

FIG. 24: A table setting forth further test results when the eye mask 100 was worn.

### MODES FOR EMBODYING THE INVENTION

Hereinafter, referring to the drawings, an explanation of preferred modes of embodying the present invention will be made. Below in the drawings, for the sake of making the explanation concise, elements and sites exhibiting the same actions are labeled with the same reference marks, and reduplicating description in some instances will be either omitted or abbreviated. Furthermore, in every diagram the dimensional relationships (length, width, thickness, etc.) in some cases do not necessarily reflect the actual dimensional relationships accurately. Nevertheless, some dimensional relationships in designated drawings have been made coincident, and in those cases, from the dimensional and positional relationships in each diagram it is possible to derive the six primary views.

Furthermore, particulars that are necessary for implementing the present invention and are matters apart from the particulars specifically referred to in the present specification can be apprehended to be particulars of design for the person skilled in the art, based on conventional technology in the given field. The present invention can be implemented based on the content disclosed by the present specification and drawings, and on common technical knowledge in the given field. What is more, the present invention is not limited to the following embodying modes.

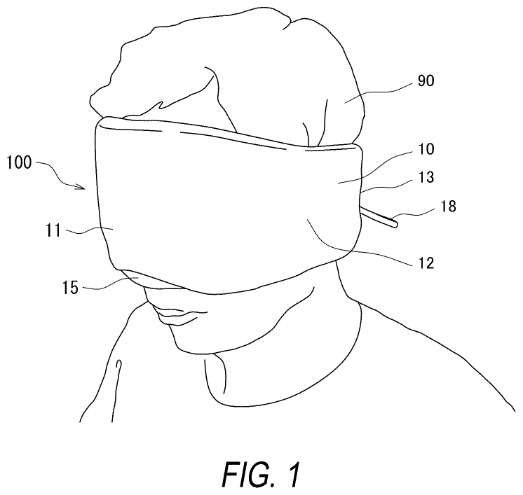

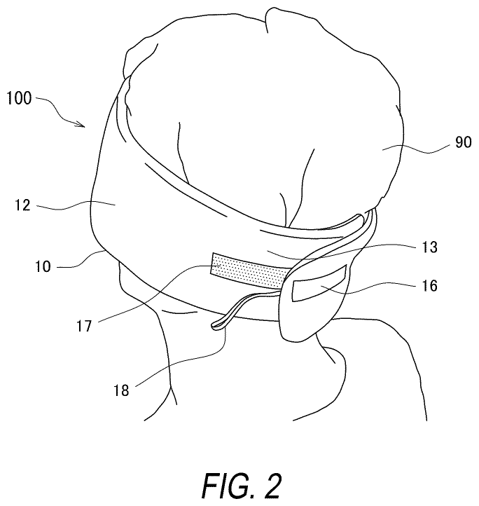

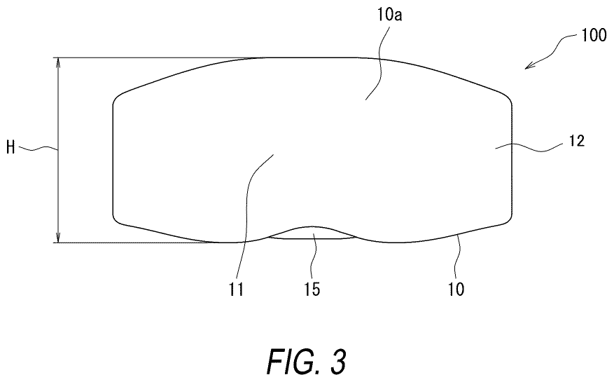

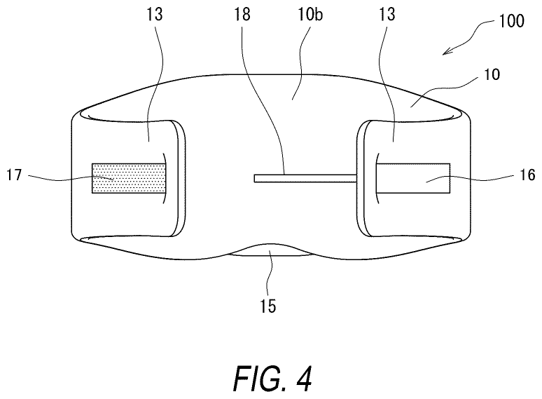

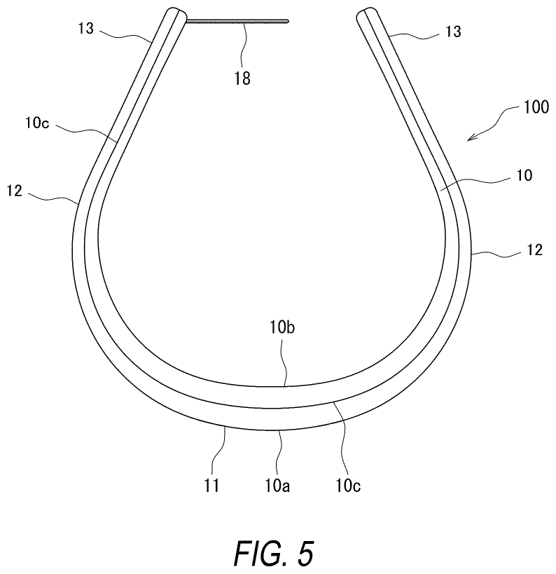

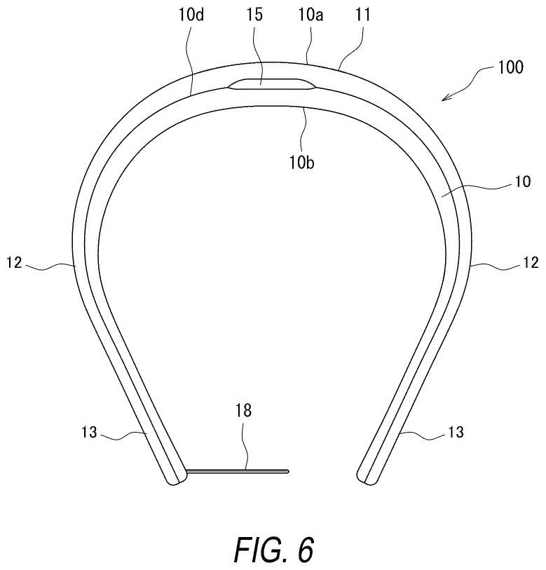

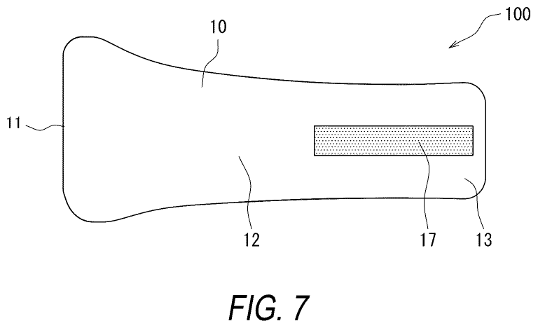

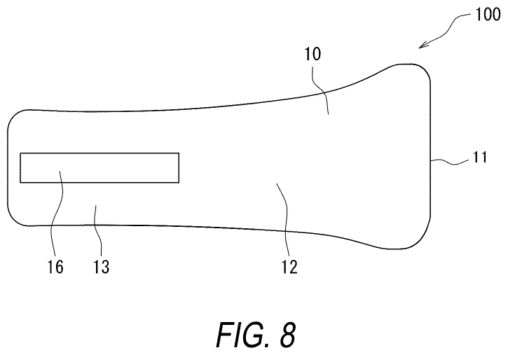

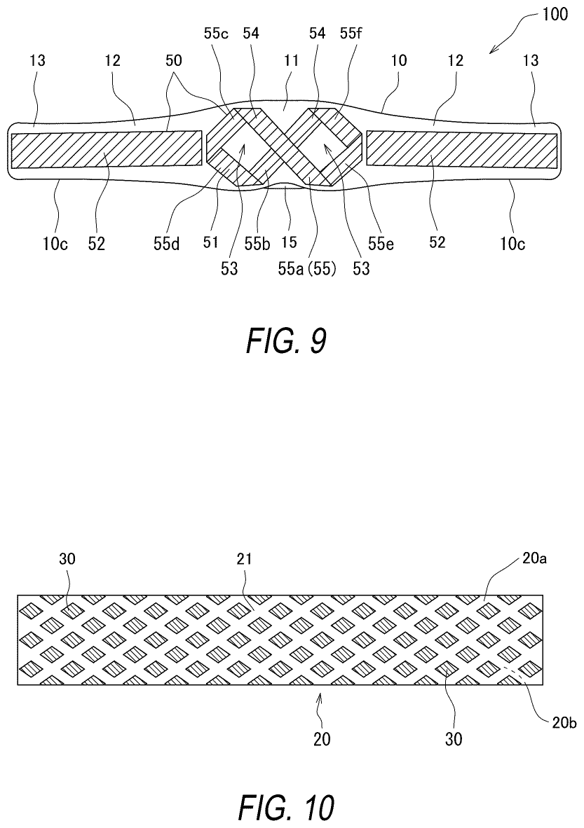

FIG. 1 is a perspective view respectively representing, along the front, the look when an eye mask 100 involving modes of embodying the present invention is worn. Likewise, FIG. 2 is a perspective view representing, along the back, the look when the eye mask 100 of the present embodying modes is worn. Furthermore, FIG. 3 through FIG. 8 respectively present a front elevation, a rear elevation, a top-side view, a bottom-side view, a right-side elevation, and a left-side elevation of the eye mask 100 of the present embodying modes. In addition, FIG. 9 is a diagram (see-through view) for explaining the configuration of mineral-containing sites 50 in the eye mask 100.

As represented particularly in FIG. 1, the eye mask 100 of the present embodying modes is furnished with an eye-mask body piece 10 that covers the eyes. Then in the eye-mask body piece 10, as represented in FIG. 9, mineral-containing sites 50 are disposed.

As indicated in FIG. 1 and FIG. 2, the eye mask 100 of the present embodying modes is fitted on a user's head 90. The eye-mask body piece 10 is made up of a frontward mask portion 11 that covers the eyes (eye area), and sideward mask portions 12 extending from the frontward mask portion 11.

With the configuration of the present embodying mode, in the lower portion of the frontward mask portion 11, a site (front under portion) 15 corresponding to the location of the nose (around the peak of the nose, or a location from under the nose to above the lips) is provided. This site (front under portion) 15, provided with a wire element (or a linear element having flexibility) on the inside of the site (e.g., inside a cloth component on the surface), enhances the fit of an eye mask 100 of the present embodying modes. It should be noted that it is not critical that this site (front under portion) 15 especially be provided.

With the configuration example of the present embodying modes, furthermore, located in sites behind the sideward mask portions 12 are mask rearward portions 13. Provided on these mask rearward portions 13 are fit-on fixing components (16 and 17), wherein by means of these fit-on fixing components (16 and 17), the eye mask 100 can be wrapped around and fixed against the head 90. The fit-on fixing components (16 and 17) of the present embodying modes are hook-and-loop fasteners (or “Magic Tape” (trade name)); by means of the hook-and-loop fasteners (16 and 17), the eye mask 100 is detachable/reattachable. The fit-on fixing components may be something else-for example, magnets (magnet-based attachment/detachment components), a zipper, buttons (including easy snap buttons), hooks, or interlocking elements enabling detachment/reattachment. With their simple structure, and given their ease of handling in use, as the fit-on fixing components, the hook-and-loop fasteners (16 and 17) are ideal. Since the hook-and-loop fasteners (16 and 17) facilitate fixing and at the same time allow easy adjustment of the tightness, users can freely make the fit they prefer. In the illustrated example, a tugger (cord) 18 is attached to the mask rearward portion 13, making it so that the eye mask 100 may be fit on comfortably.

The height (“H” in FIG. 3) of the eye mask 100 of the present embodying modes is, for example, 120 mm (±30 mm), but is not limited to that numeric figure. Likewise, the overall length of the eye mask 100 of the present embodying modes (the length when stretched out on a flat surface) is, for example, 700 mm (±100 mm), but is not limited to that numeric figure. Furthermore, in the situation in which, fixed with the hook-and-loop fasteners (16 and 17), the eye mask 100 is wrapped around the head 90, its span (diameter when made into a circle) is, for example, 200 mm ((±50 mm), but is not limited to that numeric figure. Specifically, the dimensions (height H, overall length, span (diameter)) of the eye mask 100 that are best suited to accord with its use, including for adults or for children, can be designed for/adopted.

The eye-mask body piece 10, as illustrated in FIG. 3 through FIG. 8, of the present embodying modes is constituted from an obverse-side fabric piece 10a, a reverse-side fabric piece 10b, and a boundary portion 10c therebetween. With the configuration of the present embodying modes, the obverse-side fabric piece 10a and the reverse-side fabric piece 10b are constituted from the same fabric, being a cloth component (e.g., they are constituted from synthetic-fiber cloth or natural-fiber cloth). The surface material (obverse-side fabric piece 10a and reverse-side fabric piece 10b) of the eye-mask body piece 10 of the present embodying modes may be made a mesh-type material. Having it be this sort of mesh-type material can improve breathability and, especially during the heat of summer, can improve comfort during sleep. It should be noted that for the surface material of the eye-mask body piece 10 of the present embodying modes, adopting cloth stuff that is not mesh-type would not be problematic.

In one example of the present embodying modes, the obverse-side fabric piece 10a and the reverse-side fabric piece 10b are synthetic-fiber cloth (e.g., nylon, polyurethane, or a mixture thereof), but may also be natural-fiber cloth (e.g., cotton, linen, silk, etc.). To explain further, examples that can be cited of the media (material) constituting the surface parts (obverse-side fabric piece 10a and the reverse-side fabric piece 10b) of the eye-mask body piece 10 are: synthetic-fiber textiles (e.g., nylon, polyurethane, etc.), natural-fiber textiles (e.g., cotton textiles (cottons), silk textiles (silks), etc.), mixed-yarn synthetic-fiber textiles (e.g., nylon-polyurethane blended textiles, etc.), or textiles blended from natural-fiber textiles and synthetic-fiber textiles (e.g., cotton-polyester mixed yarns, etc.), as well as blended textiles of a plurality of kinds (e.g., natural-fiber textiles (cottons, etc.)/nylon/polyurethane textiles, rayon textiles/acrylic textiles/polyester textiles, acrylic textiles/polyester textiles/rayon textiles/polyurethane textiles, etc.).

Provided in the interior of the eye-mask body piece 10 (between the obverse-side fabric piece 10a and the reverse-side fabric piece 10b) of the present embodying modes is a light-shielding material and cushioning stuff (e.g., cushioning made of urethane). Then with the configuration of the present embodying modes, provided in the interior of the eye-mask body piece 10, together with the light-shielding material and the cushioning stuff, is the mineral-containing sites 50 represented in FIG. 9.

The mineral-containing sites 50 of the present embodying modes are constituted from a frontward mineral patterning 51 that when the eye mask is worn envelops the periphery of the eyes, and sideward mineral patternings 52 that when the eye mask is worn extend along the lateral sides of the head (90). The frontward mineral patterning 51 is formed on the frontward mask portion 11 of the eye-mask body piece 10. The sideward mineral patternings 52, then, are formed on the sideward mask portions 12 of the eye-mask body piece 10.

The frontward mineral patterning 51 of the present embodying mode is configured through two openings 53, and loop sites 54 positioned on peripheries of the two openings 53. With the present embodying modes, the two openings 53 are eye-corresponding openings 53 that correspond to the eye areas. Put another way, in the frontward mineral patterning 51 are two eye-corresponding openings 53 (corresponding to the right eye and the left eye). The eye-corresponding openings 53 are configured by loop sites 54 consisting of squarish forms (approximate rhombuses, approximate squares) in a dual combination such as to envelop the eye-corresponding openings 53. With the illustrated example, the loop sites 54 have a figure-eight form (roughly the form of the number 8, a form in which an 8-like character is laid on its side). That is, the loop sites 54 assume a figure-eight form (roughly the numerical figure “8”).

It should be noted that with the configuration of the present embodying modes, since it is that of an eye mask 100 (eye-mask body piece 10) for covering the eyes, the two openings 53 are meant to be eye-corresponding openings 53; however, given that the significance of the configuration is in its figure-eight form, or in the structure of the two openings 53, the two openings 53 do not necessarily have to correspond to the right eye and the left eye.

The loop sites 54 of one example of the present embodying modes are configured combining rectilinear patterns 55 (strip sites 55a to 55f). Specifically, the loop sites 54 are formed by folding a single strand of tape material (for example, the tape material 20 depicted in FIG. 10) over itself. The single strand of tape material 20 constituting the loop sites 54 crosses over itself in a location situated in the middle between the two openings 53 (i.e., the location where the strip sites 55a and 55b cross). With the illustrated example, the strip site 55a extends atop the strip site 55b. The strip sites 55c to 55f represent those apart from the strip sites 55a and 55b, wherein together with the strip sites 55a and 55b, dual forms that are squarish (roughly rhomboidal) are created.

The length of the eye-corresponding openings 53 (the vertical or horizontal dimension of the openings) in one example of the present embodying modes is, for example, 50 mm to 80 mm (or 60 to 70 mm (±10 mm)). Not limited to being those, nevertheless, these dimensions (lengths) may, as long as beneficial effects are demonstrated, exceed the upper limit value; likewise with the lower limit value—they may fall below it.

It should be noted that the loop sites 54 of the present embodying modes are constituted as two tied-together approximate squares (approximate rhombuses), but not being limited to that, annuluses (approximate annuluses, elliptical/oval loops) or polygonal loops (loops that are polygons (approximate polygons) such as pentagons, hexagons, or octagons) may be rendered into the two tied-together loop sites 54.

Furthermore, sideward mineral patternings 52 are provided adjacent to or contiguous with the loop sites 54 of the present embodying modes. With the illustrated example, on the right side and the left side of the loop sites 54, respectively, the sideward mineral patternings 52 are provided. The sideward mineral patternings 52 in the present embodying modes are constituted from tape material 20 such as depicted in FIG. 10. Since the sideward mineral patternings 52 need only be disposed to extend along the lateral sides of the head, the sideward mineral patternings 52 need not contact the loop sites 54 and may be disposed dissociated from them. With the example illustrated in FIG. 9, the sideward mineral patternings 52 and the loop sites 54 are disposed apart. Now, where positively gaining just the function and beneficial effects of the loop sites 54 of the present embodying modes is desired, the sideward mineral patternings 52 need not be provided; but from the viewpoint of comprehensive efficacy, it is better that the sideward mineral patternings 52 be provided.

The mineral-containing sites 50 provided on the eye-mask body piece 10 of the present embodying modes are sites in which minerals are contained. The mineral-containing sites 50 of the present embodying modes are constituted from a tape material, with minerals being contained in the tape material. The tape material of the present embodying modes is constituted from cloth (cloth of natural material, or synthetic-fiber cloth), wherein either minerals are subsumed inside the tape material, or minerals are dispersed in the tape material. FIG. 10 represents one example of the tape material 20 of the present embodying modes. It should be noted that in FIG. 10, the obverse face 20a and the opposite of it, the reverse face 20b, of the tape material 20 are indicated for convenience.

To explain further, in the mineral-containing sites 50 of the present embodying modes, with one example, minerals (minerals that are known as so-called power stones (e.g., radium ore, germanium, quartz, “terahertz ore,” tourmaline, etc.), or a blend of several kinds of minerals) the particle size of which is 325 mesh (about 44 microns) are invested into the tape material 20. Also, putting in additives apart from minerals (powders) is acceptable. Minerals of at least one kind (or a blended mineral powder of two or more kinds) selected from the group consisting of radium ore, germanium, quartz, terahertz ore, and tourmaline can be utilized as the minerals (powder).

Furthermore, in situations where the tape material 20 constituting the mineral-containing sites 50 is cloth media (herein, the cotton of the backing 21 (for example, 40-count combed cotton)), it is preferable to sew on the tape material 20, affixing (anchoring) it to stuff (e.g., light-shielding cloth or cushioning) inside the eye-mask body piece 10 so that the tape material 20 does not come off or else slip out of place. What is more, it can be brought into contact with (anchored or adhered to) the internal stuff in (alternatively, the inner surface (10b) of) the eye-mask body piece 10. In that case, applying heat-sensitive adhesive (or iron-on adhesive) to the given tape material enables it to be affixed (adhesively anchored) to the eye-mask body piece 10 (the internal stuff or inner surface 10b) by heating it with a heater (herein, an iron). Likewise, anchoring it with iron-on adhesive and then sewing it in with thread is handier. It should be noted that in order to make it so that even laundered the tape material 20 will not come off or else slip out of place, it is preferable to sew on the tape material and affix (anchor) it to the eye-mask body piece 10, but it need not necessarily be done that way.

The width of the tape material 20 (tape backing 21) of the present embodying modes is, in the frontward mineral patterning 51 portion, for example 30 mm or less (with the illustrated example, e.g. 25 mm ±5 mm, or else on the order of 10 mm to 30 mm). Likewise, the width of the tape material 20 (tape backing 21) is, in the sites of the sideward mineral patternings 52, for example 70 mm or less (in the illustrated example, e.g., 50 mm±10 mm, or else on the order of 30 mm to 70 mm). The length of the tape material 20 corresponds to the frontward mineral patterning 51 and the sideward mineral patternings 52. Not limited to being those, these dimensions (the width and length of the tape material 20) may, as long as beneficial effects are demonstrated, exceed the upper limit values; likewise with the lower limit values—they may fall below them.

With the configuration example illustrated in FIG. 10, the mineral(s) contained in the tape material 20 is present as patternings 30 of design marks invested by printing. Specifically, the mineral(s) is invested by compounding into an ink a powdered mineral of at least one kind (or a blended ore powder of two or more kinds), for example, selected from the group consisting of radium ore, germanium, quartz, “terahertz” ore, and tourmaline, and by printing the mineral-containing ink on, investing it into the tape material 20 (tape backing 21). The mineral patternings 30 invested by printing have a pattern form that is geometric shapes, design marks, written characters, or a complex thereof. In the example illustrated in FIG. 10, the ore patternings 30 are squarish (e.g., rhombuses), but can be rendered patternings other than suchlike. The ore patternings 30 may be of geometric shapes such as circles (including ellipses and ovals), hexagons, or stars, or may be design marks such as waves, whirls, arabesque designs, checkered designs, floral patterns, animal patterns, landscape patterns, and cartoon/animation character patterns. The ore patternings 30 may be written characters such as alphabetic letters, numeric figures, symbols, written Japanese, Chinese characters, or Hangul. While regularly arrayed ore patternings 30, being more attractive, are preferable, it does not matter if they are in an irregular array. It should be noted that in the mineral-containing sites 50 (tape material 20) of the present embodying modes, the mineral(s) may be introduced in such a mode as to compound (or impregnate) the mineral(s) (powder) into the entirety of the tape material 20. Also, the mineral(s) (powder) may be compounded (or impregnated) into the interior (hollow) of the mineral-containing sites 50 (tape material 20) of the present embodying modes.

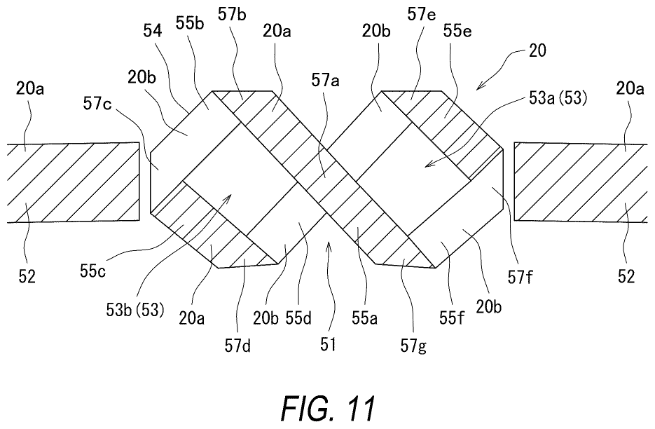

FIG. 11 illustrates, magnified, one example of a configuration of the frontward mineral patterning 51 (loop sites 54) of the present embodying modes. With the loop sites 54 illustrated in FIG. 11, by a single strand of the tape material 20 being folded over itself, sites where the obverse face 20a and the reverse face 20b of the single strand of tape material 20 overlap each other (57a to 57g) are present in at least seven locations (herein, seven locations). It should be noted that unlike what is in FIG. 9, with what is in FIG. 11, the strip sites 55a to 55f in the single strand of tape material 20 are shown such that a to f are consecutively numerical. Also, in a location (57a) situated in the middle between the two openings 53, the single strand of tape material 20 crosses over itself (the site where the strip sites 55a and 57a cross).

The minerals in the frontward mineral patterning 51 (loop sites 54) receive far-infrared heat intrinsic to the human body and generate a faint electric current (microcurrent). There a magnetic field is created. The loop sites 54 of the present embodying modes are put in a figure-eight form, and therein, the fact that in the location (57a) situated in the middle between the two openings 53, the single strand of tape material 20 crosses over itself (the site where the strip sites 55ab and 55d cross), bringing about a vector with 3D axes, can produce solid-dimensional stimulation. Put differently, the tape material is rendered into a solid-dimensional spiral structure (a solid-dimensional figure-eight structure, or solid-dimensional dual-loop structure). In instances in which the single strand of tape material 20 is not crossed over itself in the middle (i.e., instances where the structure is such that the strip sites 55ab and 55d do not cross), stimulation, turning out to be a two-dimensional reflex (a spinal reflex), even as such enters inside, yet the transmission of that stimulation proves to be indirect. That is, although there are beneficial effects even with two-dimensional reflexes (spinal reflexes), there are more beneficial effects with a structure that can bring about solid-dimensional stimulation. When the eye mask 100 is worn, a zero magnetic field is produced between the openings of the dual openings 83 of the figure-eight form, making possible the producing of a zero magnetic field in the midst of the patient's depths. Furthermore, with overlapping areas/folds in the figure eight (57a to 57g: seven locations) being present, from those locations (the seven locations) to inside (body inside, or the front side of the frontward mineral patterning 51) a cubic magnetic field directed to the center is produced. That is, a zero magnetic field in which solid-dimensional magnetic fields interfere with each other can be target-locked on the interior depths of an ailment-affected part. Figuratively, it would be a feeling that the ailment-affected part is sandwiched between the opening areas. In that the eyelid reflex (pain) works in tandem with the occipital reflex zone, pain in the eyes arises as stiffness and pain in the occipital reflex zone. The configuration of the present embodying modes can alleviate pain of that sort.

With the configuration represented in FIG. 11, while the obverse face on one end of the single strand of tape material 20 and the obverse face on the other end (conversely, the reverse face on one end of the single strand of tape material 20 and the reverse face on the other end) are connected to construct a solid-dimensional spiral structure (solid-dimensional figure-eight structure; solid intersection in an “8” figure; figure-eight spiral), a Möbius-band structure may be constructed from the single strand of tape material 20. Namely, the obverse face on one end of the single strand of tape material 20 and the reverse face on the other end (conversely, the reverse face on one end of the single strand of tape material 20 and the obverse face on the other end) may be connected to construct a Möbius-band figure-eight structure (solid-dimensional figure-eight structure of Möbius-band form). To form this sort of Möbius-band structure, if for example, in terms of the structure illustrated by FIG. 11, rather than the strip site 55a being folded back over itself and joined to the strip site 55b, a first face (e.g., the obverse face) of the strip site 55a and a second face (e.g., the reverse face) of the strip site 55b are connected, then with the single strand of tape material 20 being folded back over itself, a figure eight (a solid-dimensional figure eight) is constructed, a frontward mineral patterning 51 (loop sites 54) having the form of a Möbius band can be created. It should be noted that the locations where the obverse face of the one end of the single strand of tape material 20 is connected with the reverse face of the other end is not limited to the locations on the strip site 55a and the strip site 55b, but may be other locations. Also, a twist can be put into the single strand of tape material 20 to construct the Möbius-band structure. The frontward mineral patterning 51 (loop sites 54) being in the Möbius-band structure enables a yet more solid-dimensional magnetic field to be formed, for which reason a yet more cubic magnetic field directed toward the center proves to be produced, enabling that much more beneficial effectiveness to be gained.

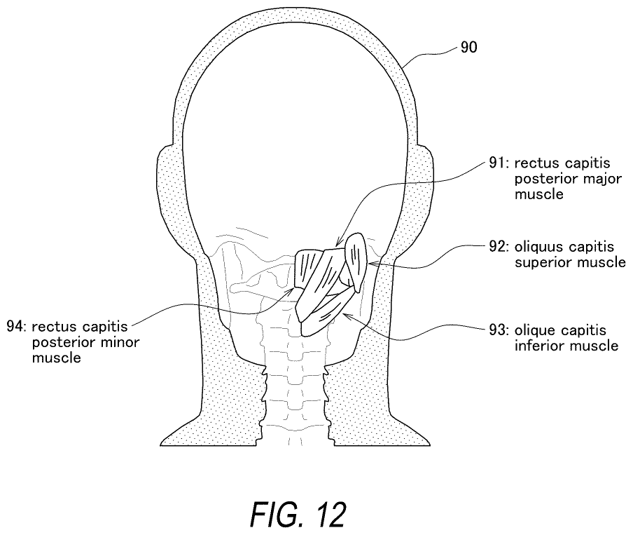

FIG. 12 schematically illustrates muscles in the nape of a head 90. The head 90 nape muscles include, for example, the rectus capitis posterior major muscle (91), the obliquus capitis superior muscle (92), the oblique capitis inferior muscle (93), and the rectus capitis posterior minor muscle (94). The eye mask 100 of the present embodying modes can influence these muscles to alleviate pain.

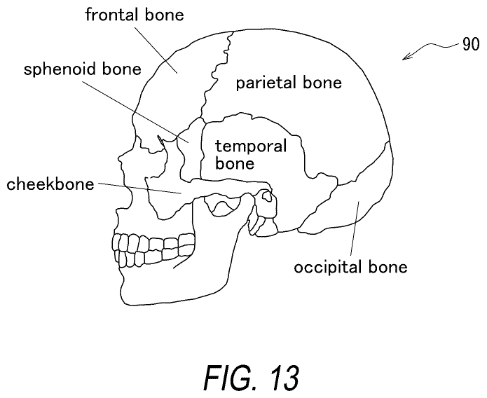

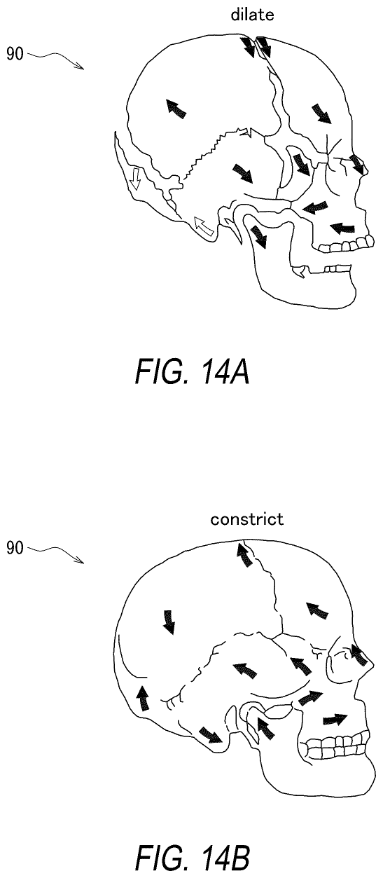

Also, together with the muscles of the head 90, the bones of the head 90 are (the skull is) also affected; as illustrated in FIG. 13, the bones of the head 90 include (the skull includes) the frontal bone, the temporal bones, the parietal bones, the occipital bone, and the cheekbones; and therein the fact that, as indicated in FIG. 14A and FIG. 14B, the skull can dilate (expand) and constrict (contract) is known.

The skull consists of the occipital bone, the sphenoid bone, the temporal bones, the frontal bone, and the parietal bones, with the entirety except for the lower cheekbones and the hyoid bone being immovably joined together. Herein, leaving aside the facial bones, to explain focusing on the braincase: in the cranium any number of sutures are present. To say that the sutures are joined immovably only amounts to a broad-strokes description; in the sutures locally they expand or contract, albeit slightly. With an eye mask 100 of the present embodying modes, being that functionality on the basis of the expansion or contraction of the sutures arises, the presence of the mineral-containing sites 50 (51 and 52) in the eye mask 100 can promote cerebrospinal fluid recirculation. Cerebrospinal fluid fills, apart from the brain interior, the spinal canal as well; and with the spinal nerve roots extending inside the canal, therein, that an eye mask 100 of the present embodying modes can improve athletic functioning (physical functioning and flexibility besides) is because it can make neurotransmission fluid in the pyramidal tracts and extrapyramidal system.

Herein, the explanation will be continued further on the premise that the human body can produce smooth movement by means of the muscles as a pseudo-fluid structure. This smooth movement also is in that there is movement of the human skeleton. Skeletal movement is based on structural leaning pressure owing to one's center-of-balance base of support compensating with respect to movement from above. Herein the expansion/constriction (contraction) of the skull works in tandem with movement of the diaphragm (muscle), meaning that it is in synchronization with breathing. By the inhaling and exhaling of breath, the diaphragm moves; then, naturally, by the working of the muscles in tandem the inclination of the skeleton changes, whereby the skull moves. To explain further: when with breathing the diaphragm moves, the ilium moves in tandem and in response to the breathing a front turn (the ilium curls inward) or a back turn (the ilium opens outward) takes place. Then in response to the pelvic movement, the skull expands or contracts its sutures, carrying out fine adjustments to gain gravitational-center balance. Movement along the opposite route also naturally exists. Consequently, the skull shifts the sutures albeit slightly. With healthy movement, when the pelvis (the ilium particularly) opens, the skull contracts, while when the pelvis (the ilium particularly) closes, the skull enlarges. With unhealthy movement, when the pelvis and skull take on a rhythm in which in synchronization they expand/contract at the same point, exercise/physiology-wise various complaints arise of vague malaise. The human body through breathing is constantly making subtle movements (especially, in this case movements of the skull and ilium). An eye mask 100 of the present embodying modes, then, can modulate this movement to what feels right.

An eye mask 100 of the present embodying modes is furnished with mineral-containing sites 50 (51 and 52); the presence of these mineral-containing sites 50 (51 and 52) can promote cerebrospinal fluid recirculation and also modulate the skull then the ilium to make the gravitational-center balance into what feels right, and thereby can alleviate fatigue in a wearer (user) wearing the eye mask 100 and further, can improve physical functioning and flexibility besides. It should be noted that while “taping,” in which tape is stretched directly on the body—often used especially by athletes—works to protect muscles, with the technology of the present embodying modes gentle stimulation—in contrast to strong stimulation from affixing tape directly—is availed of, and as it were, thus may be referred to as “noncontact-model indirect taping.” It is supposed, then, that thanks to properties of the mineral-containing sites 50 (51 and 52), given regions can be activated, alleviating fatigue in the wearer (user), and exercise capacity can be assisted. It should be noted that though these properties may be slight, in a biological phenomenon, effective stimulation of given locations can take place, accomplishing exercise-capacity improving functions; that is (inasmuch as the body is a cluster of delicate sensors), it being enough that they occasion stimulation, even if the difference in properties due to the presence or absence of the mineral-containing sites 50 (51 and 52) is slight, beneficial effects of alleviating fatigue and improving exercise capacity can be demonstrated. And like it is a fact that acupuncture and moxibustion performed on the head can have an effect on the body, an eye mask 100 of the present embodying modes, simply by being worn on the head, can alleviate fatigue in the wearer, and improve physical functioning and flexibility besides. Also, the possibility that by minerals being compounded into the tape material 20, influences from the minerals (e.g., negative ions, terahertz waves, static electricity, far infrared rays, hormesis, etc.) amount to stimuli is conjectured, and the possibility that the beneficial effects from an eye mask 100 of the present embodying modes are thereby obtained is conjectured. It should be noted that with an eye mask 100 of the present embodying modes, as opposed to making the entirety of the head 90 mineral-containing sites 50, from the viewpoint of noncontact-model indirect taping (or from a view toward cranial-suture vectors and stimulation zones), it is preferable that regions where mineral-containing sites 50 are not present (e.g., openings 53 (eye-corresponding openings 53)) exist. To explain further: Comprehensively synthesizing such viewpoints, the mineral-containing sites 50 (51 and 52) in an eye mask 100 of the present embodying modes are constructed.

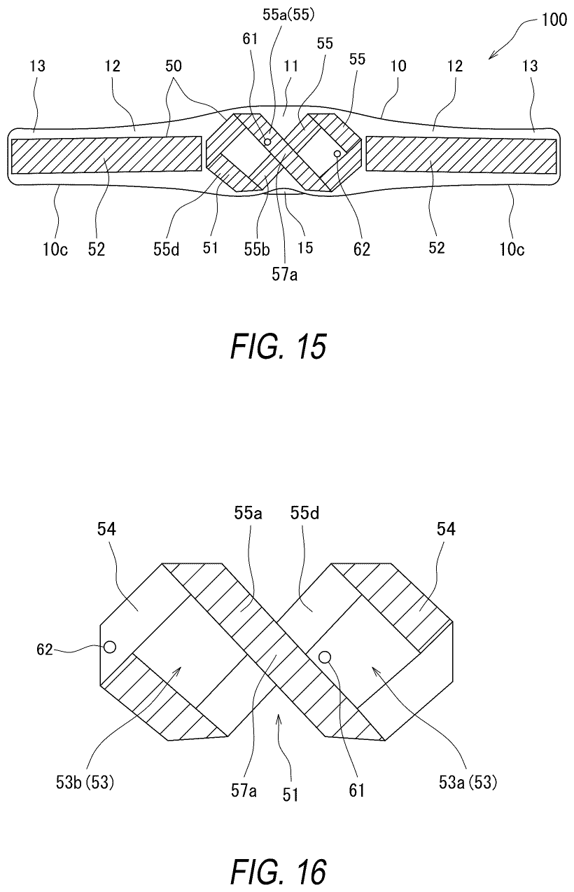

Next, as illustrated in FIG. 15 modifications to an eye mask 100 of the present embodying modes can be carried out. FIG. 15 schematically represents the configuration of mineral-containing sites 50 (51 and 52) in a modified example of an eye mask 100 of the present embodying modes. On the frontward mineral patterning 51 (herein, the regions of the loop sites 54) of the mineral-containing sites 50 represented in FIG. 15, a first patch 61 constituted from a first metal (in this case, kin (gold)) is formed. Then, by the frontward mineral patterning 51 (herein, in the region of an eye-corresponding opening 53), a second patch 62 constituted from a second metal (in this case, gin (silver)) is formed. It will be appreciated that FIG. 16 is matched to the structure represented in FIG. 11. In the structure represented in FIG. 16, the first patch 61 and the second patch 52 may be arranged. In FIG. 16, the first patch 61 is provided in the region of the right loop portion (i.e., the site where the right-side opening 53a is present). The second patch 52 is provided in the region of the left loop portion (i.e., the site where the left-side opening 53b is present).

The first patch 61 (gold patch) constituted from a first metal (in this case, kin (gold), or else a substance having a golden metal at least on the surface), and the second patch 62 (silver patch) constituted from a second metal (in this case, gin (silver), or else a substance having a silvery metal at least on the surface) also can be arranged in the following way. At first providing the first patch (gold patch) in at least a part of the loop sites 54 and the openings 53 (53a) that corresponds to one of either the right eye or the left eye (in this case, the right eye), then providing the second patch 62 (silver patch) in at least a part of the loop sites 54 and the openings 53 (53b) that corresponds to the other than the one of either the right eye or the left eye (in this case, the left eye) is preferable. It is permissible, however, that they stick out slightly outside the loop sites 54 (for example, it is permissible that the second patch 62 (silver patch) be shifted slightly leftward in the plane of the drawing sheet, as long as it is in the vicinity of the loop site 54).

With the configuration of the present embodying modes, as indicated in FIG. 16, the first patch (gold patch) 61 is provided in a loop site 54, then the second patch (silver patch) 62 is provided in at least one region (53a) of the two openings 53. With other configurational examples, both the first patch (gold patch) 61 and the second patch (silver patch) 62 are disposed atop the loop sites 54. To give one example: The first patch (gold patch) 61, in terms of the example depicted in FIG. 16, is disposed on the loop site 54 in a position where it is shifted to the upper left in the drawing-sheet plane (with the example depicted in FIG. 11, the area on the strip site 55d equivalent to the drawing-sheet plane upper right). The second patch (silver patch) 62 is for instance positioned in the location of the example depicted in FIG. 16. In this case, the first patch 61 and the second patch 62 can be sewn on to anchor them to the loop sites 54. In instances of configurations in which, by being sewn with thread, the loop sites 54 are anchored to the frontward mask portion 11, inasmuch as the first patch 61 and the second patch 62 can be sewn on and anchored fast by means of the thread, manufacturing operations-wise, as a method of anchoring the first patch 61 and the second patch 62 it is simple and has considerable advantages. It will be appreciated that anchoring the first patch 61 and the second patch 62 to the loop sites 54 with anchoring means separate from thread for fixing (e.g., yarn, adhesive, etc.) is not a problem.

Furnishing only a portion of the first patch (gold patch) 61 (e.g., 30% to 70% (with one example, 50%)) on a loop site 54 is, further still, acceptable. Then furnishing only a portion of the second patch (silver patch) 62 (e.g., 30% to 70% (with one example, 50%)) on a loop site 54 is acceptable. Likewise, both the first patch 61 and the second patch 62—only portions thereof (e.g., 30% to 70% (with one example, 50%))—may be disposed atop the loop sites 54. Put another way, the embodying mode may be such that the first patch (gold patch) 61 and/or the second patch (silver patch) 62 is disposed atop a boundary (outer border or inner border) of the loop sites 54. Disposing the first patch (gold patch) 61 and/or the second patch 62 on a boundary of the loop sites 54 (that is, a borderline of the tape material (e.g., a borderline of the tape material 20 represented in FIG. 10)) yields an interference waveform; that operates, in showing compensating movement, to give sustaining the special features (beneficial effects) of the figure eight. As a single preferred disposition example, with the configuration depicted in FIG. 16, the first patch (gold patch) 61 is shifted to the drawing-sheet plane upper left to dispose it on the boundary of the strip site 55d. For example, it is disposed in such a way that half (approximately half) of the first patch 61 is placed on the strip site 55d. Likewise, the second patch (silver patch) 62 is shifted to the drawing-sheet plane left to dispose it on the left-edge boundary of the strip site. For example, it is disposed in such a way that half (approximately half) of the second patch 62 is placed on the strip site. Placement in these ways is one preferred disposition example.

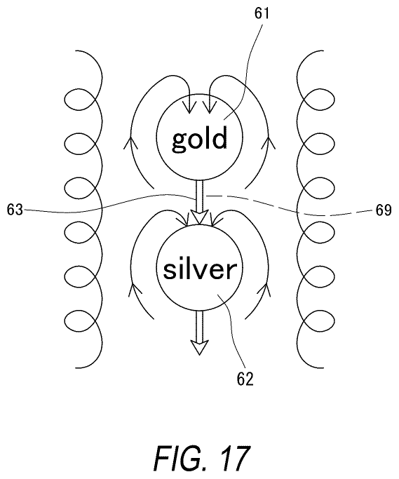

FIG. 17 is a schematic diagram for explaining the relationship between the first patch (gold patch) 61 and the second patch (silver patch) 62. Herein, in that faint electric currents flow through the human body, around the human body magnetic fields are generated. With the configuration of the present embodying mode, as indicated by arrow 69, free electrons are induced (or else biophotons are generated) through the first patch (gold patch) 61 and led toward the second patch (silver patch) 62, and a magnetic field (arrow 63) from the first patch (gold patch) 61 into the second patch (silver patch) 62 is formed, which can modulate the human body's magnetic fields. The first patch (gold patch) 61 is, speaking from a magnetic viewpoint, tantamount to the S-pole of a magnet; likewise, the second patch (silver patch) 62 is, speaking from a magnetic viewpoint, tantamount to the N-pole of a magnet. In the region of the interval from the first patch (gold patch) 61 to the second patch (silver patch) 62, a region of substantially zero magnetic field arises as an interference wave between the N-pole and the S-pole. This interference wave (zero-magnetic-field region) being brought about is desirable since, with a three-dimensional-axis vector being formed, it leads to promotion of cerebrospinal fluid recirculation taking place. Herein, the potential difference between the first patch (61) and the second patch (62) (the potential difference between heterogeneous metals—e.g., between brass and/or bronze, and aluminum) gives rise to a flow of ions whose functionality is exploited. Put in terms, then, of the relationship between the gold patch 61 and the silver patch 62: given that from wavelengths of gold and silver colors the physical characteristics reflection and absorption are exhibited, the fact that gold/silver light colors (electromagnetic waves) possess the properties of N-poles/S-poles is exploited to lend the patches the functionality, with the configuration of the present embodying mode, of markings (triggers) rendered for giving rise to free electrons (or biophotons).

With the example configuration of the present embodying mode, the first patch (gold patch) 61 and the second patch (silver patch) 62 are rendered circular, but they may be made other shapes (e.g., elliptical, oblong, squarish, hexagonal, etc.). And the number first patches (gold patches) 61 and second patches (silver patches) 62 that are best suited can be adopted. Furthermore, the example configuration of the present embodying mode has the first patch 61 be a gold patch and has the second patch 62 be a silver patch, but inasmuch as the configuration is not thereby limited, the patches can be made pairings of other, differing kinds of metals. Such a pairing of differing kinds of metals is one that generates a magnetic flow (arrow 63, etc.) from the first patch 61 to the second patch 62 when an eye mask 100 of the present embodying mode is worn on a head 90. As an example of this sort of pairing of differing kinds metals, a pairing of aluminum (tantamount to the N-pole) and a metal consisting of zinc/copper (tantamount to the S-pole) can be given. Likewise, a pairing of yellow copper (an alloy of copper and zinc) and aluminum (100% aluminum metal) can be given. Others that can be cited are a pairing of nickel-yellow copper (an alloy of copper, zinc, and nickel) and aluminum, a pairing of cupronickel (an alloy with copper as the core component and containing from 10% to 30% nickel) and aluminum, and a pairing of copper and aluminum. To explain further: In the configuration of the present embodying mode, the first patch 61 has been described as being a gold patch and the second patch 62 as being a silver patch, but the first patch 61 may be a metal selected from the group consisting of metals composed of at least one of either zinc or copper, yellow copper, nickel-yellow copper, and cupronickel. Likewise, the second patch 62 may be a metal composed of aluminum. In addition, in one example of the present embodying mode, the first patch 61 (for example, a gold patch) is a metal-coated golden thin-film article, while the second patch 62 (for example, a silver patch) is a metal-coated silvery thin-film article. The metal coating can be carried out by means of vacuum deposition (thin-film forming technology). Vacuum deposition techniques that can be utilized include physical vapor deposition (PVD), ion assisted deposition, and chemical vapor deposition. Alternatively, rather than by vacuum deposition, the metal coating may be carried out by plating (metal plating). In addition, rather than metal coating, metal films/metal sheets/metal layers (e.g., aluminum foil or the like as the second patch 62) can be utilized to form the first patch 61/second patch 62. In the forming of the first patch 61/second patch 62 by vacuum deposition (metal vapor deposition), a structure in which onto a substrate-constituting base-film layer (for example, a PVC film [a polymer resin film with the principal component being polyvinyl chloride (PVC)], a polyester film [e.g., a PET film (polyethylene terephthalate film)], or the like) a vapor deposition layer (metal vapor deposition layer) has been deposited may be fabricated, and the layered structure processed into the geometry of the first patch 61/second patch 62. A layered structure may be fabricated by laminating (bonding or the like) a metal film/metal foil (metal layer) onto another article (a base layer), and the laminated structure processed into the geometry of the first patch 61/second patch 62. Inasmuch as the structure will function as a first patch 61/second patch 62 as long as metal is contained in the surface, the first patch 61/second patch 62 may be created with the entirety being a metal material, or metal may be imparted to the surface by coating (e.g., vacuum deposition, plating, lamination/thin-film formation) to create the first patch 61/second patch 62. As simple, convenient patches utilizing films, sequined articles (thin-film articles having a metallic color—what is used on clothing, for example) may be acceptably employed.

With an eye mask 100 of the present embodying modes, the two openings 53 and the loop sites 54 are formed in the frontward mineral patterning 51 among the mineral-containing sites 50 disposed on the eye-mask body piece 10 that covers the eyes; thereby, when the eye mask 100 is worn, a zero magnetic field is produced between the two openings 53, making for a situation where the zero magnetic field can reach the central depths of an ailment-affected part, which as a result can alleviate fatigue in the wearer.

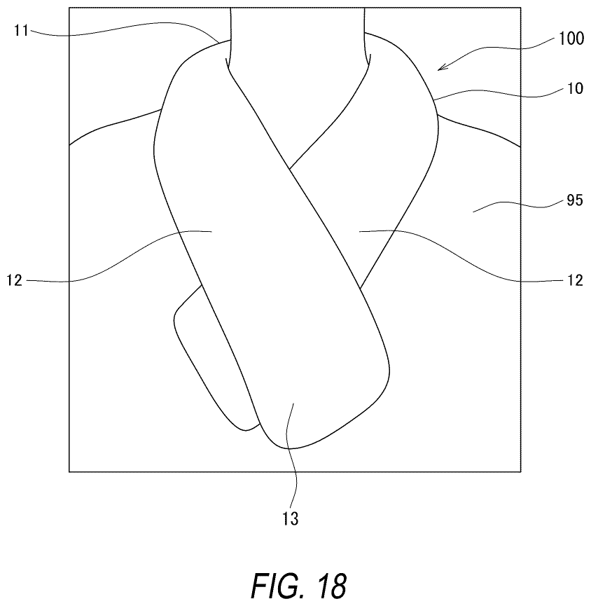

It should be noted that an eye mask 100 of the present embodying modes not only can be worn on the head 90 such as to cover the eyes, but also can be wrapped around the nape of the user's (employer's) 95 neck, as represented in FIG. 18. Putting it in this way also can yield at least some of the benefits of an eye mask 100 of the present embodying modes (including elevating body temperature and alleviating neck pain, for example).

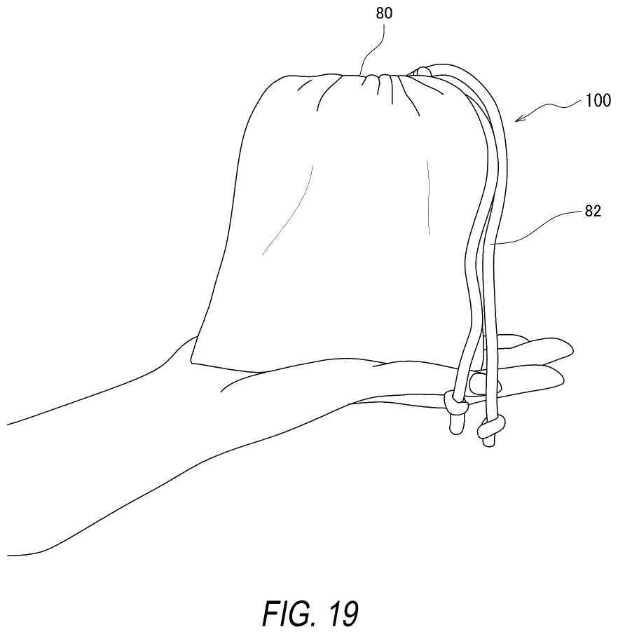

What is more, an eye mask 100 of the present embodying modes can be put in a storage bag 80, as represented in FIG. 19. In this way being able to put an eye mask 100 of the present embodying modes into a compact mode makes carrying it around handy, and convenient especially on the go (including airplane, train, ship, and car travel), in that it can be used at once. The storage bag 80 is provided with a cord 82, which is convenient in that the mouth of the storage bag 80 can be drawn shut with the cord 82, and at the same time the cord 82 can be hung somewhere.

### EMBODYING EXAMPLES

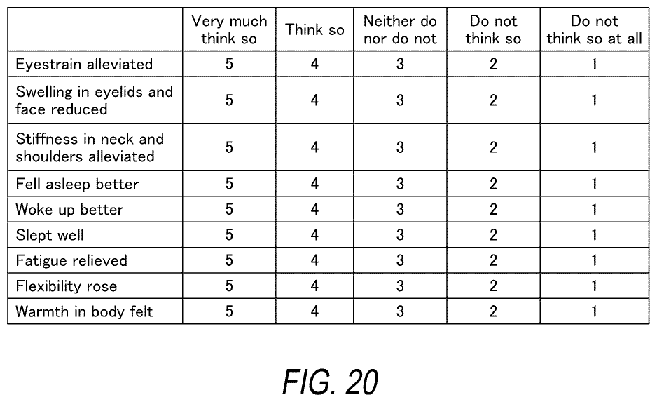

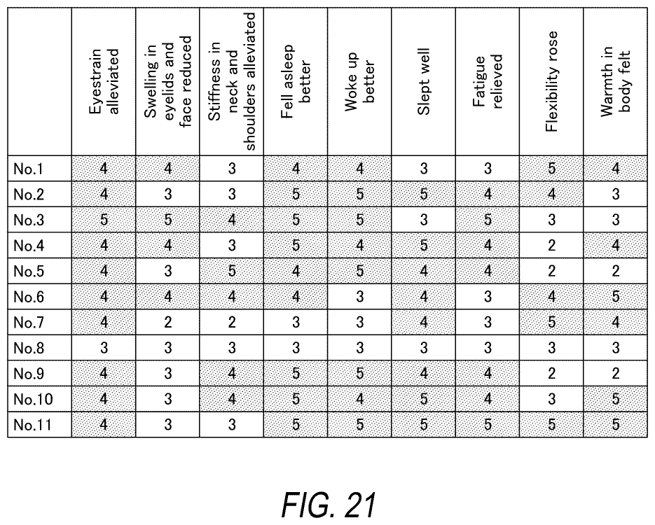

Next, the beneficial effects (effectiveness tests) of embodying examples of instances in which an eye mask 100 of the present embodying modes was worn will be specifically described. FIG. 20 sets forth testing indicators for when the eye mask 100 was worn, and FIG. 21 sets forth test results corresponding to the testing indicators.

As presented in FIG. 21, with embodying examples (effectiveness tests) of instances in which the eye mask 100 was worn, the indicators “eyestrain alleviated,” “swelling in eyelids and face reduced,” “stiffness in neck and shoulders alleviated,” “fell asleep better,” “woke up better,” “slept well,” “fatigue relieved,” “flexibility rose,” and “warmth in the body felt” were the effectiveness-test items, with the evaluation being five-grade, from “5” for “very much think so” to “1” for “do not think so at all.” The results for 11 persons as test subjects were, in the different evaluations, “4's” and “5's” totaling numerously, with do-not-think-so “2's” being few, and do-not-think-so-at-all “1's” not being present. The beneficial effects of an eye mask 100 of the present embodying modes were thereby experimentally confirmed.

Also, upon tests, separate from those tests, for checking flexibility being performed, significant betterment in pre-/post-wear an eye mask 100 of the present embodying modes with “forward bends,” “rearward arm swings,” etc. was gained. This will be discussed later.

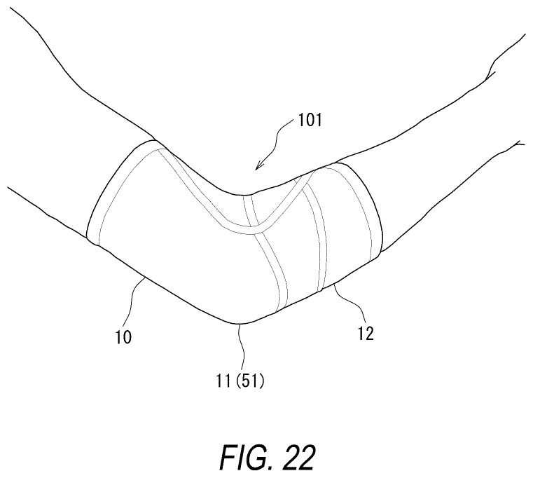

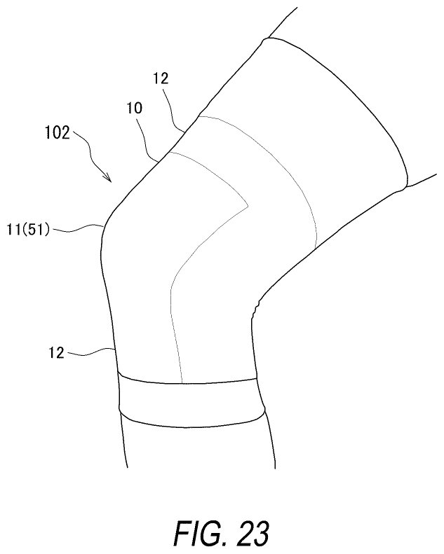

With the above-described embodying modes, while modes of an eye mask 100 have been described, given that beneficial effects whereby elbow and knee pain are alleviated can be obtained, using it as a corporeal contact article (including as a support article) is possible. Specifically, a corporeal contact article of the present embodying modes can be used also as supports (101 and 102) on sites including the elbows and knees. In FIG. 22 is a support 101 for use on the elbow, while in FIG. 23 is a support 102 for use on the knee. Onto a site where pain occurs (e.g., an elbow or a knee), they are wrapped in such a way as to make the loop sites 54 (the figure-eight site) of the mineral-containing sites 50, depicted in FIG. 9 (or FIG. 11, FIG. 15, or FIG. 16), be applied. The loop sites 54 (figure-eight site) of the mineral-containing sites 50 can, as described above, give rise to vectors with axes in three dimensions, producing solid-dimensional stimulation (releasing energy solid-dimensionally), mitigating pain in the location concerned.

In situations in which the structure of the present embodying modes is employed as corporeal contact articles (e.g., supports 101 and 102), the corporeal contact articles (101 and 102) of the present embodying modes are constituted from a corporeal-contact body section (support body section) 10 for covering a portion of the body (e.g., knee, elbow, etc.), and mineral-containing sites 50 (in particular, frontward mineral patternings 51 (53 and 54)) situated in the corporeal-contact body section (support body section) 10. The support body section 10 is constituted from a frontward support portion (ailment-affected part contacting part) 11 for covering a portion of the body (e.g., knee, elbow, etc.) and support sideward portions 12 extending from the frontward support portion 11. On the frontward support portion 11, among the mineral-containing sites 50 a frontward mineral patterning 51 (component in figure-eight form) for covering a portion of the body (e.g., knee, elbow, etc.) is formed, wherein on the sideward support portions 12 of the support body section 10, sideward mineral patternings 52 are formed. This sort of support (e.g., 101 and 102) can be exploited. It should be noted that while the sideward mineral patternings 52 need not be provided, providing the sideward mineral patternings 52 can make the comprehensive effectiveness greater. It will be appreciated that with FIG. 22 and FIG. 23, although the sideward mineral patternings 52 are provided longitudinally (in the direction along which the arms and legs extend), they may also be provided along a circuiting direction (direction wrapping around the arms/legs).

A corporeal contact article of the present embodying modes may furthermore be, in addition to an item that directly contacts a knee, elbow, or other site, an item that is put in indirect contact (or near contact) with the body. Specifically, the mineral patterning 51 (component in figure-eight form (reference is made to FIG. 16, for example) or Möbius-band component) can be employed as a corporeal contact article (corporeal near-contact article). As described above, given that in the mineral patterning 51, the loop sites 54 (figure-eight site) of the mineral-containing sites 50, bringing about a vector with 3D axes, can produce solid-dimensional stimulation (may release energy solid-dimensionally), even if they are not directly in contact, if they are directly in contact (if they are in near-contact) advantageous effects can be demonstrated. As a utilization example of this sort, the frontward mineral patterning 51 (component in figure-eight form, Möbius-band component) can be disposed, by for example putting the article inside a necktie, to make it brought into near contact with the body. Likewise, the mineral patterning 51 may be utilized by making it into a necklace of sorts (an article that is part of a necklace). In other ways as well—the mineral patterning 51 can be utilized by making it into an accessory (e.g., part of a key holder, part of jewelry, etc.).

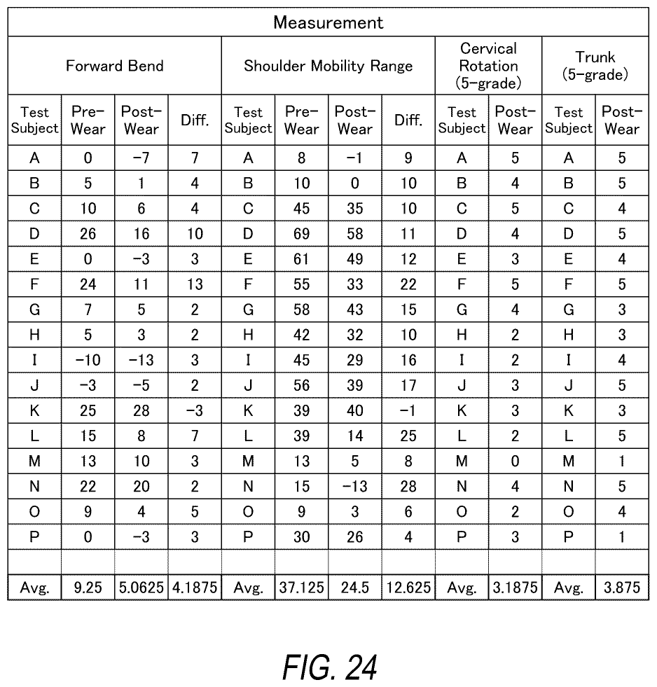

In addition, further beneficial effects of embodying examples (effectiveness tests) of instances in which an eye mask 100 of the present embodying modes was worn will be specifically described. Wearing an eye mask 100 of the present embodying modes serves also in betterment of physical functioning (especially in improvement of flexibility), and this beneficial effect will be described. FIG. 24 presents results in which betterment of physical functioning was measured in situations in which the eye mask 100 was worn.

With the effectiveness tests, “forward bend,” “shoulder mobility range” (rearward arm swing), “cervical rotation” and “trunk” (improvement in core strength) were conducted. The test subjects were 16 persons, A to P, and in trials of each individual's forward bend and shoulder mobility range, pre-wear and post-wear numerical values were measured. In the cervical rotation and trunk trials, change in the individual was self-evaluated in five grades, with 5 being greatest presence of effectiveness, 3 being ordinary, and 1 being the greatest absence of effectiveness.

Given that with cervical rotation, change in numerical value will itself be slight (while even 1 cm is a significant change, the numerical value is small), self-evaluation is better suited to revealing effectiveness. Since better than a numerical figure for a weighted change, the person themselves may best have a real sense of trunk (improvement in core strength), here too self-evaluation is better suited to revealing effectiveness.

In this effectiveness trial a platform to which was attached a measuring rule, with the platform surface being 0 cm, of 1-cm intervals from above and below it was prepared, and this was utilized to measure “forward bend” (cm), a body-erect forward bend. With the forward bend, what was +9.2 cm pre-wear, became +5.0 cm post-wear, wherein a betterment of 4.2 cm was seen. With the person for whom there was the most betterment (test subject F) their forward bend bettered a good 13 cm simply by them wearing an eye mask 100.

For “shoulder mobility range” (cm), the test subject stood with both arms spread out straight to the sides, then another person lightly grasped the test subject's wrists, pulled them to the rear, checked how far to the rear the hands reached, and measured the distance separating the two hands along the subject's back. With shoulder mobility range, what was 37.1 cm pre-wear, became 24.5 cm post-wear, wherein a betterment of 12.6 cm was seen. With the person for whom there was the most betterment (test subject N), their shoulder mobility range improved a good 28 cm simply by them wearing an eye mask 100 (post-wear, the subject's hands crossed a good 13 cm).

“Cervical rotation” (five-grade) was carried out by the neck being rotated horizontally from side to side. With cervical rotation bettering on average a good 3.1 with pre-/post-wear, it was recognized that there was effectiveness with neck rotatability (improvement in neck flexibility).

“Trunk” (improvement in core strength) was carried out with the test subject folding the fingers on either hand together and placing them in front of (and next behind) the body, and another person placing their fists on top of, and putting their weight on, the test subject. Trunk axial tremor having been felt in the front (then the back) of the body pre-/post-wear was evaluated. The rating 5 was for the greatest lack of having felt axial tremor, while the rating 1 was for greatest lack of having felt effectiveness. With trunk bettering on average a good 3.8 in pre-/post-wear, it was recognized that there was effectiveness in core-strength improvement.

In this way, it was confirmed that the wearing of an eye mask 100 of the present embodying modes serves in betterment of physical functioning (especially, in improvement of flexibility).

While in the foregoing, the present invention has been described according to preferred embodying modes, the description thus rendered is not a limiting matter; inarguably various modifications are possible. The above-described embodying modes as well as modified-example configurations as well as techniques are applicable to one another. An eye-mask body piece 10 (eye mask 100) of the present embodying modes is designed to be used while sleeping (napping included), but may also be used during intervals or breaks in sports (e.g., golf, tennis, soccer, baseball, etc.) or at work or in studying for exams. Also, although a structure of the present embodying modes was employed as an elbow or knee support, there is nothing prohibiting its use in locations apart from those, as a support (or as a mode other than a support, including therapeutic devices, pain-alleviating devices, fatigue-reducing devices, stress-reducing devices, and devices that aid in maintaining (increasing) concentration). Likewise, while the mineral-containing sites 50 (including the frontward mineral patterning 51) were rendered covered with cloth media (a lining 10b) or the like, if the issue of the feeling when put on (wearing comfort) is ignored, it would not matter if cloth media (lining 10b) of this sort is omitted. What is more, other suitable modifications can be made as appropriate.

### INDUSTRIAL EXPLOITABILITY

According to the present invention, an eye mask that alleviates fatigue in the wearer can be made available.

### DESCRIPTION OF REFERENCE MARKS

10: eye-mask body piece (corporeal-contact body section, support body section
11: frontward mask portion
12: sideward mask portion
13: mask rearward portion
20: tape material (tape article)
30: ore patterning
50: mineral-containing site
51: frontward mineral patterning
52: sideward mineral patterning
53: opening
53: (eye-corresponding opening)
54: loop site
55a to 55f: strip sites
100: eye mask
101: corporeal contact article (elbow support)
102: corporeal contact article (knee support)

1. An eye mask comprising:
an eye-mask body piece for covering eyes; and
mineral-containing sites situated in the eye-mask body piece and containing a mineral;
the eye mask being wherein
the eye-mask body piece is constituted from
a frontward mask portion for covering the eyes, and
sideward mask portions extending from the frontward mask portion;
on the frontward mask portion of the eye-mask body piece, among the mineral-containing sites a frontward mineral patterning for enveloping the eyes' periphery is formed;
the frontward mineral patterning is configured through two openings, and loop sites positioned on peripheries of the two openings; and
the loop sites are created by a single strand of tape material being folded over itself.

2. The eye mask set forth in claim 1, wherein the loop sites have a figure-eight form.

3. The eye mask set forth in claim 2, wherein the single strand of tape material constituting the loop sites crosses over itself in a location situated midway between the two openings.

4. The eye mask set forth in claim 2, wherein in the loop sites, by the single strand of tape material being folded over itself, sites where obverse and reverse faces of the single strand of tape material overlap each other are present in at least seven locations.

5. The eye mask set forth in claim 1, wherein the loop sites are of figure-eight form having a Möbius-band structure.

6. The eye mask set forth in claim 1, wherein the two openings in the frontward mineral patterning are eye-corresponding openings that correspond to eye areas.

7. The eye mask set forth in claim 1, wherein on the eye-mask body piece a first patch constituted from a first metal is formed, and wherein separated from the first patch, a second patch constituted from a second metal is formed.

8. The eye mask set forth in claim 7, wherein the first patch is a gold patch, and therein the second patch is a silver patch.

9. The eye mask set forth in claim 7, wherein:
the two openings in the frontward mineral patterning are eye-corresponding openings that correspond to eye areas;
the first patch is provided on at least a part of the loop sites or the openings that is one of either right-eye or left-eye correspondent;
the second patch is provided on at least a part of the loop sites and the openings that is the other than the one of either right-eye or left-eye correspondent.

10. The eye mask set forth in claim 7, wherein:
the first patch is provided in the loop sites in the frontward mineral patterning; and
the second patch is provided in at least one region of the two openings in the frontward mineral patterning.

11. The eye mask set forth in claim 7, wherein:
the first patch is provided in the loop sites in the frontward mineral patterning; and
the second patch is provided in the loop sites in the frontward mineral patterning.

12. The eye mask set forth in claim 11, wherein the first patch and the second patch are sewn on, anchoring them to the loop sites.

13. The eye mask set forth in claim 11, wherein only a portion of the first patch is provided on the loop sites.

14. The eye mask set forth in claim 11, wherein only a portion of the second patch is provided on the loop sites.

15. The eye mask set forth in claim 1, wherein on the sideward mask portions of the eye-mask body piece, among the mineral-containing sites sideward mineral patternings for extending along a head's lateral sides are formed.

16. The eye mask set forth in claim 15, wherein the sideward mineral patternings are made up of rectilinear patterns.

17. The eye mask set forth in claim 1, wherein:
the mineral is at least one type, or a mixture of a plurality of mineral types, selected from the group consisting of radium ore, germanium, quartz, terahertz ore, and tourmaline;
the mineral is invested into the tape material by being printed onto it; and
the mineral invested by the printing have a pattern form selected from the group consisting of geometric shapes, design marks, and written characters.

18. The eye mask set forth in claim 1, wherein the mineral-containing sites are disposed interiorly in the eye-mask body piece.

19. An eye mask comprising:
an eye-mask body piece for covering eyes; and
mineral-containing sites situated in the eye-mask body piece and containing a mineral(s);
the eye mask being wherein
the eye-mask body piece is constituted from
a frontward mask portion for covering the eyes, and
sideward mask portions extending from the frontward mask portion;
on the frontward mask portion of the eye-mask body piece, among the mineral-containing sites a frontward mineral patterning for enveloping the eyes' periphery is formed;
the frontward mineral patterning is configured through two openings, and loop sites positioned on peripheries of the two openings; and
on the eye-mask body piece a first patch constituted from a first metal is formed, wherein separated from the first patch a second patch constituted from a second metal is formed.

20. The eye mask set forth in claim 19, wherein the first patch is a gold patch, and therein the second patch is a silver patch.

21. The eye mask set forth in claim 19, wherein:
the two openings in the frontward mineral patterning are eye-corresponding openings that correspond to eye areas;
the first patch is provided on at least a part of the loop sites or the openings that is one of either right-eye or left-eye-correspondent;
the second patch is provided on at least a part of the loop sites and the openings that is the other than the one of either right-eye or left-eye correspondent.

22-27. (canceled)

28. A corporeal contact article comprising:
a corporeal-contact body section for covering a portion of the body; and
mineral-containing sites situated in the corporeal-contact body section and containing a mineral;
the corporeal contact article being wherein
the mineral-containing sites are configured through two openings, and loop sites positioned on peripheries of the two openings; and
the loop sites are created by a single strand of tape material being folded over itself.

29. The corporeal contact article set forth in claim 28, wherein the loop sites have a figure-eight form.

30. The corporeal contact article set forth in claim 29, wherein the single strand of tape material constituting the loop sites crosses over itself in a location situated midway between the two openings.

31. The corporeal contact article set forth in claim 28, wherein in the loop sites, by the single strand of tape material being folded over itself, sites where obverse and reverse faces of the single strand of tape material overlap each other are present in at least seven locations.

32. The corporeal contact article set forth in claim 28, wherein the loop sites are of figure-eight form having a Möbius-band structure.

33. The corporeal contact article set forth in claim 28, wherein:
the mineral is at least one type, or a mixture of a plurality of mineral types, selected from the group consisting of radium ore, germanium, quartz, terahertz ore, and tourmaline;
the mineral is invested into the tape material by being printed onto it; and
the mineral invested by the printing have a pattern form selected from the group consisting of geometric shapes, design marks, and written characters.

34. The corporeal contact article set forth in claim 28, wherein on the mineral-containing sites a first patch constituted from a first metal is formed, wherein separated from the first patch a second patch constituted from a second metal is formed.

35. The corporeal contact article set forth in claim 28, wherein for the mineral-containing sites, further, extension mineral patternings that extend adjacent to or contiguous with the loop sites are provided.

36. The corporeal contact article set forth in claim 28, wherein the corporeal contact article is a support article for protecting at least one of an elbow or a knee.

37. The corporeal contact article set forth in claim 28, wherein the corporeal contact article is disposed within a necktie.
